(19) United States
(12) Patent Application Publication
(10) Pub. No.: US 2023/0255909 A1
(43) Pub. Date: Aug. 17, 2023

(54) METHODS OF ADMINISTERING DOFETILIDE

(71) Applicant: AltaThera Pharmaceuticals LLC, Chicago, IL (US)

(72) Inventor: Brandon Ira Kashfian, Chicago, IL (US)

(21) Appl. No.: 18/306,660

(22) Filed: Apr. 25, 2023

Related U.S. Application Data
(63) Continuation-in-part of application No. 17/892,301, filed on Aug. 22, 2022.
Application No. 17/892,301 is a continuation-in-part of application No. 17/566,840, filed on Dec. 31, 2021, now Pat. No. 11,610,660.
(60) Provisional application No. 63/276,947, filed on Nov. 8, 2021.
(60) Provisional application No. 63/235,500, filed on Aug. 20, 2021.
(60) Provisional application No. 63/345,068, filed on May 24, 2022.
(60) Provisional application No. 63/344,154, filed on May 20, 2022.
(60) Provisional application No. 63/340,581, filed on May 11, 2022.
(60) Provisional application No. 63/334,267, filed on Apr. 25, 2022.
(60) Provisional application No. 63/331,905, filed on Apr. 18, 2022.
(60) Provisional application No. 63/276,947, filed on Nov. 8, 2021.
(60) Provisional application No. 63/235,500, filed on Aug. 20, 2021.
(60) Provisional application No. 63/345,068, filed on May 24, 2022.
(60) Provisional application No. 63/344,154, filed on May 20, 2022.
(60) Provisional application No. 63/340,581, filed on May 11, 2022.
(60) Provisional application No. 63/334,267, filed on Apr. 25, 2022.

(51) Int. Cl. A61K 31/18; A61K 9/00; A61P 9/06

(52) U.S. Cl. CPC A61K 31/18; A61K 9/0019; A61P 9/06

(57) ABSTRACT

Methods of administering dofetilide in an amount effective for treating a cardiovascular condition are described. An IV dose can be administered over a selected period of time, such as about one hour, followed by a first oral dose. The patient can be discharged from the medical facility providing cardiac monitoring prior to administration of additional oral doses. The IV and first oral doses can be administered in a manner such that maximum serum concentration of dofetilide is reached and the patient can be discharged within about 7-8 hours.

### CROSS-REFERENCE TO RELATED APPLICATIONS

This application is a Continuation-in-Part (CIP) application of U.S. application Ser. No. 17/892,301 filed Aug. 22, 2022. The '301 application is a Continuation-in-Part application of U.S. application Ser. No. 17/566,840 filed Dec. 31, 2021, which application relies on the disclosure of and claims priority to and the benefit of the filing date of each of U.S. Provisional Application Nos. 63/235,500 filed Aug. 20, 2021 and 63/276,947 filed Nov. 8, 2021. The '301 application relies on the disclosure of and claims priority to and the benefit of the filing date of each of U.S. Provisional Application Nos. 63/235,500 filed Aug. 20, 2021, 63/276,947 filed Nov. 8, 2021, 63/331,905 filed Apr. 18, 2022, 63/334,267 filed Apr. 25, 2022, 63/340,581 filed May 11, 2022, 63/344,154 filed May 20, 2022, and 63/345,068 filed May 24, 2022. The present application relies on the disclosure of and claims priority to and the benefit of the filing date of each of U.S. Provisional Application Nos. 63/334,267 filed Apr. 25, 2022, 63/340,581 filed May 11, 2022, 63/344,154 filed May 20, 2022, and 63/345,068 filed May 24, 2022. The disclosures of these applications are hereby incorporated by reference herein in their entireties.

### FIELD OF THE INVENTION

The present disclosure is directed to the field of cardiovascular pharmaceutics, more particularly compositions and methods for the treatment of cardiovascular conditions such as arrhythmias with intravenous anti-arrhythmics, such as dofetilide.

### BACKGROUND

Dofetilide is a class III antiarrhythmic agent which acts through blocking cardiac ion channels of the rapid component of the delayed rectifier potassium current Ikr. The agent, a sulfonamide, is approved to treat atrial fibrillation and atrial flutter. Dofetilide normalizes sinus rhythm by prolonging cardiac action potential duration and effective refractory period due to delayed repolarization without affecting conduction velocity. Dofetilide is currently approved in the US for oral administration under the brand name TIKOSYN® (Pfizer Inc., New York, N.Y.). It is not approved for parenteral administration.

### SUMMARY OF THE INVENTION

Described herein are methods of administering dofetilide to a subject, such as a patient in need thereof, in an amount effective for treating a cardiovascular condition of the patient. In embodiments, the effective amount of dofetilide can be administered intravenously as a loading dose of 0.1 to 12 μg/kg bodyweight over a duration for example of up to about 60 minutes. In embodiments, one or more maintenance dose(s) is/are given orally and/or infused intravenously as an IV maintenance dose of 0.1 to 10 μg/kg/hr, such as over a duration of at least 1 hour.

In embodiments, the cardiovascular condition can include atrial fibrillation, atrial flutter, ventricular tachycardia, hemodynamically stable or unstable ventricular tachycardia, ventricular fibrillation, paroxysmal supraventricular tachycardia, paroxysmal atrial fibrillation, heart failure, coronary artery disease, pulmonary artery hypertension, atrial tachycardia, junctional ectopic tachycardia, or junctional tachycardia. The method can further include measuring a QT interval (or QTc) of the patient before, during, and/or after the administering, and selecting or adjusting the effective amount, the loading dose and/or the maintenance dose based on the QT interval or QTc at any point during administering the protocol. The method can further include measuring a creatinine clearance of the patient before the administering, and selecting or adjusting the effective amount, the loading dose and/or the maintenance dose based on the creatinine clearance.

Aspects of embodiments of the invention include Aspect 1, which is a method, comprising: administering dofetilide to a subject, such as a patient; wherein the dofetilide is administered: as a loading dose, intravenously, over a period of about 1 hour; and as a first IV or oral maintenance dose, in an amount of 125 μg, 250 μg, or 500 μg, wherein optionally the IV dosage, and the first IV or oral maintenance dose, are administered to the subject while admitted to a facility capable of providing cardiac resuscitation and continuous electrocardiographic monitoring and subsequent IV or oral maintenance doses are administered to the subject after having been discharged from such facility, or the IV dosage is administered to the subject while admitted to a facility capable of providing cardiac resuscitation and continuous electrocardiographic monitoring and the first IV or oral maintenance dose and any subsequent oral doses are administered to the subject after having been discharged from such facility, optionally wherein a prescription is provided for administering any of the first IV or oral maintenance dose and/or any subsequent IV or oral maintenance dose.

Aspect 2 is a method of administering dofetilide comprising: administering an IV loading dosage of dofetilide to a subject; wherein the IV dosage is administered over a period of about 1 hour; wherein the subject is in a facility capable of providing cardiac resuscitation and electrocardiographic monitoring; and wherein the IV dosage is administered in an amount of about 62.5-750 μg; and after administering the IV dosage, administering an oral dosage of dofetilide to the subject in an amount of 125 μg, 250 μg or 500 μg, wherein optionally the oral dosage is also administered to the subject while admitted to the facility capable of providing cardiac resuscitation and continuous electrocardiographic monitoring and any subsequent oral doses are administered to the subject after having been discharged from such facility, or the oral dosage and any subsequent oral doses are administered to the subject after having been discharged from such facility, optionally wherein a prescription is provided for administering any of the oral dosages or doses.

Aspect 3 is the method of Aspect 1 or 2, further comprising repeating oral administration of dofetilide at least once at a selected interval.

Aspect 4 is the method of any of Aspects 1-3, wherein the repeating of the oral administration is performed after the subject is discharged from the facility capable of providing cardiac resuscitation and electrocardiographic monitoring.

Aspect 5 is the method of any of Aspects 1-4, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, paroxysmal supraventricular tachycardia, junctional ectopic tachycardia, junctional tachycardia, heart failure, coronary artery disease, and pulmonary artery hypertension.

Aspect 6 is the method of any of Aspects 1-5, wherein the subject is currently in sinus rhythm and/or has experienced cardioversion to sinus rhythm.

Aspect 7 is the method of any of Aspects 1-6, wherein: the IV dosage is administered in an amount of: (a) about 62.5-187.5 μg; (b) about 125-375 μg; or (c) about 250-750 μg; and the oral dosage is administered in an amount of: (a) 125 μg after completion of the (a) IV dosage; or (b) 250 μg after completion of the (2)(b) IV dosage; or (c) 500 μg after completion of the (2)(c) IV dosage.

Aspect 8 is the method of any of Aspects 1-7, wherein the IV dosage is administered in an amount of: (a) about 62.5-137.5 wherein the subject is being initiated for an oral dose of dofetilide in the amount of 125 (b) about 125-275 wherein the subject is being initiated for or escalated to an oral dose of dofetilide in the amount of 250 μg; or (c) about 250-550 wherein the subject is being initiated for or escalated to an oral dose of dofetilide in the amount of 500 μg.

Aspect 9 is the method of any of Aspects 1-8, wherein the oral dosage is administered to the subject starting at least about 2 hours after completion of the IV dosage.

Aspect 10 is the method of any of Aspects 1-9, where in the IV dosage is administered as a single IV dosage.

Aspect 11 is the method of any of aspects 1-10, where in the IV dosage is administered in an amount that is +50% of the oral dosage amount, ±30% of the oral dosage amount, ±25% of the oral dosage amount, ±20% of the oral dosage amount, or ±10% of the oral dosage amount.

Aspect 12 is the method of any of Aspects 1-11, wherein: the subject is being initiated or escalated to a higher dose of oral dofetilide; the administering comprises: determining kidney function, such as a creatinine clearance, of the subject; determining a QTc interval of the subject; administering to the subject an IV loading dose of dofetilide, wherein the IV loading dose is selected from an amount ranging from 0.1-12 μg/kg, and determining a second QTc interval of the subject; administering one or more oral dose(s) of dofetilide to the subject; optionally determining one or more subsequent QTc interval of the subject between one or more of the oral doses; and optionally administering one or more subsequent oral doses to the subject wherein the oral dose administered is selected from 125 μg, 250 μg, or 500 μg, wherein optionally the IV dosage and the first oral dose are administered to the subject while admitted to a facility capable of providing cardiac resuscitation and continuous electrocardiographic monitoring and subsequent oral doses are administered to the subject after having been discharged from such facility, or the IV dosage is administered to the subject while admitted to a facility capable of providing cardiac resuscitation and continuous electrocardiographic monitoring and the first and any subsequent oral doses are administered to the subject after having been discharged from such facility, optionally wherein a prescription is provided for administering any of the oral dosages or doses.

Aspect 13 is the method of any of Aspects 1-12, wherein the IV loading dose is administered over about 60 minutes, optionally by way of several IV doses.

Aspect 14 is the method of any of Aspects 1-13, wherein the administering of the oral dose(s) begins about 1-12 hours after completion of the administering of the IV loading dose, such as beginning about 2-4 hours after, or about 4-6 hours after, or about 6-8 hours after, or about 8-10 hours after, or about 10-12 hours after.

Aspect 15 is the method of any of Aspects 1-14, wherein the administering of the oral dose(s) occurs at a 12-hour, 24-hour or 48-hour interval from a previous oral dose.

Aspects 16 is the method of any of Aspects 1-15, wherein a second oral dose of dofetilide is administered to the subject about 12-48 hours after the first oral dose.

Aspect 17 is the method of any of Aspects 1-16, wherein the IV loading or maintenance dose is administered in an amount of up to 750 μg, or up to 550 μg, or up to 300 μg, or up to 150 μg.

Aspect 18 is the method of any of Aspects 1-17, wherein the subject had received a prior dose of dofetilide 12-24 hours prior to administration of the IV loading dose.

Aspect 19 is the method of any of Aspects 1-18, wherein the subject is capable of experiencing a dofetilide Cₘₐₓ steady state within about 8 hours of administration of the IV loading dose, such as within about 4 hours, or within about 2 hours, or within about 1 hour.

Aspect 20 is the method of any of Aspects 1-19, wherein the IV loading dose and the first oral dose are administered to the subject in a facility capable of providing cardiac resuscitation and EKG monitoring.

Aspect 21 is the method of any of Aspects 1-20, wherein the second oral dose and/or subsequent oral dose(s) are administered after the subject is discharged from the facility capable of providing cardiac resuscitation and EKG monitoring.

Aspect 22 is the method of any of Aspects 1-21, wherein the subject has a creatinine clearance of >60 mL/min before being administered the IV dofetilide.

Aspect 23 is the method of any of Aspects 1-21, wherein the subject has a creatinine clearance of 40 mL/min to 60 mL/min.

Aspects 24 is the method of any of Aspects 1-21, wherein the subject has a creatinine clearance of 20 mL/min to <40 mL/min.

Aspect 25 is the method of any of Aspects 1-24, wherein the subject is discharged with instructions to administer subsequent oral doses of dofetilide every 12 to 48 hours.

Aspect 26 is the method of any of Aspects 1-25, further comprising measuring a QT or QTc interval of the subject before administration of the IV loading dose.

Aspect 27 is the method of any of Aspects 1-26, further comprising measuring a QT or QTc interval of the subject after administration of the IV loading dose and before the first oral dose.

Aspect 28 is the method of any of Aspects 1-27, further comprising measuring a QT or QTc interval of the subject after administration of the second oral dose or any subsequent oral dose.

Aspect 29 is the method of any of Aspects 1-28, wherein the second oral dose amount is different from the first oral dose amount, such as the second oral dose being a lower amount.

Aspect 30 is the method of any of Aspects 1-29, wherein the subject is being treated for atrial fibrillation and/or atrial flutter, and optionally is in sinus rhythm.

Aspect 31 is the method of any of Aspects 1-30, wherein the IV loading dose is administered to the subject in an amount and over a period of time such that the dofetilide reaches or is capable of reaching a Cₘₐₓ in the subject that is at least about 70% of a steady state Cₘₐₓ for an oral dosing protocol of 125 μg, 250 μg, or 500 μg of dofetilide, such as about 75%, 80%, 85%, 87%, 90%, 92%, 95%, 97% or 99%.

Aspect 32 is a method of administering dofetilide comprising: administering to a subject an intravenous (IV) dosage of dofetilide, wherein the subject is in a facility capable of providing cardiac resuscitation and continuous electrocardiographic monitoring, and wherein the IV dosage is administered: (a) in an amount of about 62.5-187.5 μg; (b) in an amount of about 125-375 μg; or (c) in an amount of about 250-750 μg; and after completion of the administering of the IV dosage, administering a maintenance dose of dofetilide; optionally administering a subsequent maintenance dose of dofetilide at least once at a selected interval, optionally after the subject is discharged from the facility capable of providing cardiac resuscitation and continuous electrocardiographic monitoring.

Aspect 33 is the method of Aspect 32, wherein the IV dosage of dofetilide and the maintenance dose are administered to the subject in a facility capable of providing cardiac resuscitation and continuous electrocardiographic monitoring, and the subsequent maintenance dose of dofetilide, if administered, is administered after the subject is discharged from the facility capable of providing cardiac resuscitation and continuous electrocardiographic monitoring.

Aspect 34 is the method of Aspect 32 or 33, wherein the IV dosage of dofetilide is administered as a single IV dosage over a period of about 1-5 hours, and the maintenance dose of dofetilide is administered at least 1 hour after completion of the administration of the IV dosage of dofetilide.

Aspect 35 is the method of any of Aspects 32-34, wherein: the maintenance dose of dofetilide is an intravenous maintenance dose and/or an oral maintenance dose; and the subsequent maintenance dose of dofetilide, if administered, is a subsequent intravenous maintenance dose and/or a subsequent oral maintenance dose comprising an amount of dofetilide that is the same or a different amount of dofetilide as in the maintenance dose.

Aspect 36 is the method of any of Aspects 32-35, wherein the maintenance dose and/or the subsequent maintenance dose, if administered, comprises an amount of dofetilide that is less than the IV dosage, and/or the subsequent maintenance dose, if administered, comprises an amount of dofetilide that is less than the maintenance dose.

Aspect 37 is the method of any of Aspects 32-36, wherein the IV dosage comprises dofetilide in an amount in the range of about 50% to about 110% of the amount of dofetilide in the maintenance dose.

Aspect 38 is the method of any of Aspects 32-37, wherein the maintenance dose and/or the subsequent maintenance dose, if administered, is an oral maintenance dose of dofetilide in an amount of 125 μg, 250 μg, or 500 μg.

Aspect 39 is the method of any of Aspects 32-38, wherein the maintenance dose and/or the subsequent maintenance dose, if administered, is an intravenous maintenance dose of dofetilide in an amount in the range of about 62.5 μg-750 μg.

Aspect 40 is the method of any of Aspects 32-39, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

Aspect 41 is the method of any of Aspects 32-40, further comprising: measuring a baseline QT interval and/or determining a baseline QTc interval for the subject before administering the IV dosage; determining one or more QT interval(s) or QTc interval(s) after administration of the maintenance dose; if at least one of the one or more QT or QTc intervals determined after the maintenance dose is more than 500 msec or increases greater than 15% from the baseline QT or QTc interval, refraining from administering a subsequent maintenance dose of dofetilide.

Aspect 42 is the method of any of Aspects 32-41, wherein the IV dosage of dofetilide is administered: in a first higher amount for the subject who is renally impaired; and in a second lower amount for the subject who is non-renally impaired.

Aspect 43 is the method of any of Aspects 32-42, wherein the amount of dofetilide for any one or more or all doses is below 1.8 μg/kg.

Aspect 44 is the method of any of Aspects 32-43, wherein the amount of dofetilide for any one or more or all doses is above 3 μg/kg.

Aspect 45 is the method of any of Aspects 32-44, wherein the amount of dofetilide for any one or more or all doses is an amount in the range of about: 85-100 μg, 90-150 μg, 105-115 μg, 175-200 μg, 190-300 μg, 210-240 μg, 400-450 μg, 425-550 μg, or 440-475 μg.

Aspect 46 is the method of any of Aspects 32-45, wherein the IV dosage comprises an amount of dofetilide based on a creatinine clearance of the subject as follows:

| | Intravenous Dosage of Dofetilide | | |
|---|---|---|---|
| | | | Subject's |
| Maintenance | Subject's | Subject's | CrCl 20 mL/min |
| Dose | CrCl >60 mL/min | CrCl 40-60 mL/min | to <40 mL/min |
| 125 μg | 85-100 μg | 90-150 μg | 105-115 μg |
| 250 μg | 175-200 μg | 190-300 μg | 210-240 μg |
| 500 μg | 400-450 μg | 425-550 μg | 440-475 μg |

Aspect 47 is a method of administering dofetilide, comprising: before administering an IV dosage of dofetilide to a subject, determining a creatinine clearance (CrCl) of the subject, and selecting an amount of dofetilide to administer to the subject based on the creatinine clearance; administering to the subject the IV dosage of dofetilide comprising the selected amount of dofetilide based on the creatinine clearance and comprising an amount of dofetilide in the range of 0.1-12 μg/kg, and after the administering of the IV dosage, administering a first maintenance dose of dofetilide to the subject; optionally administering one or more subsequent maintenance doses of dofetilide at selected interval from the first maintenance dose.

Aspect 48 is the method of Aspect 47, wherein the IV dosage and the first maintenance dose of dofetilide are administered to the subject in a facility capable of providing cardiac resuscitation and continuous electrocardiographic monitoring, and the subsequent maintenance dose of dofetilide, if administered, is administered after the subject is discharged from the facility capable of providing cardiac resuscitation and continuous electrocardiographic monitoring.

Aspect 49 is the method of Aspect 47 or 48, wherein the first maintenance dose of dofetilide is an intravenous maintenance dose and/or an oral maintenance dose.

Aspect 50 is the method of any of Aspects 47-49, wherein: the first maintenance dose and/or one or more of the subsequent maintenance doses, if administered, of dofetilide is a higher dose than the IV dosage.

Aspect 51 is the method of any of Aspects 47-50, wherein: the first maintenance dose and/or one or more of the subsequent maintenance doses, if administered, of dofetilide is a lower dose than the IV dosage.

Aspect 52 is the method of any of Aspects 47-51, wherein the IV dosage comprises an amount of dofetilide in the range of about ±50% of the amount of the first maintenance dose.

Aspect 53 is the method of Aspects 47-52, wherein the IV dosage is administered over about 60 minutes.

Aspect 54 is the method of any of Aspects 47-53, wherein the subject is capable of experiencing a dofetilide Cₘₐₓ steady state within about 8 hours of administration of the IV dosage.

Aspect 55 is the method of any of Aspects 47-54, wherein the IV dosage is administered to the subject in an amount and over a period of time such that the dofetilide reaches or is capable of reaching a Cₘₐₓ in the subject that is at least about 70% of a steady state Cₘₐₓ for an oral dosing protocol of 125 μg, 250 μg, or 500 μg dofetilide.

Aspect 56 is the method of any of Aspects 47-55, wherein the IV dosage of dofetilide is administered: in a first higher amount for the subject with a CrCl of 20 mL/min to <40 mL/min; and in a second lower amount for the subject with a CrCl of 40 mL/min or greater.

### DETAILED DESCRIPTION OF VARIOUS EMBODIMENTS OF THE INVENTION

Reference will now be made in detail to various illustrative implementations. It is to be understood that the following discussion of the implementations is not intended to be limiting.

AF is atrial fibrillation.

AFL is atrial flutter.

AF/AFL=atrial fibrillation and/or atrial flutter.

IV is intravenous.

PO means “per os” and refers to an oral dosing regimen.

BID means “bis in die” and means twice a day.

QD means “quaque die” and means once a day.

QID means “quater in die” and means four times a day.

Patient (or subject) refers to a human subject or patient.

BP is blood pressure.

HR is heart rate.

Renally impaired refers to patients/subjects having creatinine clearance rates of ≤60 mL/min, such as ≤30 mL/min.

Cₘₐₓ ss is the maximal concentration obtained at steady state.

QT is the interval measured from the start of the Q wave or the QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQT is the difference between a QT measurement taken prior to the start of treatment and a QT measured after the start of treatment (e.g., during loading or maintenance).

ΔQTc is the difference between a QTc measurement taken prior to the start of treatment and a QTc measured after the start of treatment (e.g., during loading or maintenance).

The terms “treat,” “treating,” and “treatment” refer to any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's physical or mental well-being. The treatment or amelioration of symptoms can be based on objective or subjective parameters, including the results of a physical examination, neuropsychiatric examinations, or psychiatric evaluation.

Hospital refers to a medical facility staffed and equipped to provide continuous ECG monitoring and cardiac resuscitation to patients, if needed. Typically, the medical personnel are trained in the management of serious ventricular arrhythmias.

Escalation means increasing the dofetilide dosage of a patient already receiving dofetilide, for example where a subject is currently taking a specific amount of an oral dose and escalation involves administering one or more IV doses to escalate the subject to a higher target oral dose.

The term “about” used herein in the context of quantitative measurements means the indicated amount ±10%. For example, “about 2 mg” can mean 1.8-2.2 mg.

IV and Oral Dosing

According to embodiments of the invention, the dofetilide can be administered intravenously as a loading dose in the range of 0.1 to 8 μg/kg, including 0.1, 0.2, 0.3, 0.4, 0.5, 0.6, 0.7, 0.8, 0.9, 1.0, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.2, 4.4, 4.5, 4.6, 4.8, 5.0, 5.2, 5.4, 5.5, 5.6, 5.8, 6.0, 6.2, 6.4, 6.5, 6.6, 6.8, 7.0, 7.2, 7.4, 7.5, 7.6, 7.8, 8.0, μg/kg, or in any range with any of these values as lower or upper values, such as 1.5 to 4.4 μg/kg, 3.2 to 6.8 μg/kg, 2.6 to 5.4 μg/kg, 2.0 to 6.0 μg/kg, 1.8 to 4.8 μg/kg, and so on. Higher loading doses, such as those exceeding 8.0 μg/kg, such as 8.2 to 12.0 μg/kg, including 8.2, 8.4, 8.5, 8.6, 8.8, 9.0, 9.2, 9.4, 9.5, 9.6, 9.8, 10.0, 10.2, 10.4, 10.5, 10.6, 10.8, 11.0, 11.2, 11.4, 11.5, 11.6, 11.8, and 12.0 μg/kg, or in any range with any of these values as lower and upper values, such as 8.2 to 10 μg/kg may be possible, such as, if administered over longer durations.

In embodiments, the loading dose amount is selected from an amount up to about ±50% of a target oral dose or a target maintenance dose, such as up to about ±5%, ±10%, ±15%, ±20%, ±25%, ±30%, ±35%, ±40%, or ±45%. In embodiments, for a target oral dose of 500 μg, the loading dose amount may be in the range of about 250 μg to about 750 μg, such as about 275 μg to about 725 μg, about 300 μg to about 700 μg, about 325 μg to about 675 μg, about 350 μg to about 650 μg, about 375 μg to about 625 μg, about 400 μg to about 600 μg, about 425 μg to about 575 μg, about 450 μg to about 550 μg, or about 475 μg to about 525 μg. In embodiments, for a target oral dose of 250 μg, the loading dose may be in the range of about 125 μg to about 375 μg, such as about 137.5 μg to about 362.5 μg, about 150 μg to about 350 μg, about 162.5 to about 337.5 μg, about 175 μg to about 325 μg, about 187.5 μg to about 312.5 μg, about 200 μg to about 300 μg, about 212.5 μg to about 287.5 μg, about 225 μg to about 275 μg, or about 237.5 μg to about 262.5 μg. In embodiments, for a target oral dose of 125 μg, the loading dose may be in the range of about 62.5 μg to about 187.5 μg, such as about 68.75 μg to about 181.25 μg, about 75 μg to about 175 μg, about 81.25 μg to about 168.75 μg, about 87.5 μg to about 162.5 μg, about 93.75 μg to about 156.25 μg, about 100 μg to about 150, about 106.25 μg to about 143.75 μg, about 112.5 μg to about 137.5 μg, or about 118.75 μg to about 131.25 μg.

In embodiments, the IV loading dose is administered while the patient/subject is admitted to a hospital or other facility capable of providing cardiac resuscitation and continuous electrocardiographic monitoring. The loading dose can be slowly titrated upward until restoration of normal sinus rhythm as measured by electrocardiogram (ECG), or ceased or adjusted downward if ventricular arrhythmias, ventricular tachycardia, or other side effects of dofetilide, such as dizziness, are observed. In embodiments, the loading dose can be adjusted for patient QT interval or QTc as described below. In embodiments, the loading dose can be adjusted for patient creatinine clearance as described below.

In embodiments, the IV loading dose is delivered by way of an IV bolus or infusion over a period of about 60 minutes, such as about 0.5, 1, 2, 3, 4, 5, 6, 7, 8, 9, 10, 11, 12, 13, 14, 15, 20, 25, 30, 35, 40, 45, 50, 55, 60, 65, or 70 minutes, or within any range with any of these values as lower and upper values, such as 5 to 20 minutes, 10 to 40 minutes, 15 to 30 minutes, 20 to 45 minutes, 30 to 60 minutes, 35 to 50 minutes, 35 to 60 minutes, 40 to 60 minutes, 45 to 60 minutes, and so on.

The loading dose can be intravenously infused into a central or peripheral vein of the patient. The flow rate of the intravenous dose can be adjusted based on the concentration of the dofetilide in the intravenous formulation, the desired duration of administration, and the body weight of the patient to achieve a specific loading dose. Thus, longer administration durations of a specific dose (X μg/kg or mg/kg) will typically have slower flow rates compared to shorter administration durations to achieve the same dose.

According to some embodiments, after the loading dose, the dofetilide can be infused intravenously as a maintenance dose at a rate in the range of 0.1 to 10 μg/kg/hr, including 0.1, 0.2, 0.3, 0.4, 0.5, 0.6, 0.7, 0.8, 0.9, 1.0, 1.2, 1.3, 1.4, 1.5, 1.6, 1.8, 1.9. 2.0, 2.2, 2.4, 2.5, 2.6, 2.7, 2.8, 3.0, 3.1, 3.2, 3.3, 3.4, 3.5, 3.6, 3.8, 4.0, 4.2, 4.3, 4.4, 4.5, 4.6, 4.7, 4.8, 5.0, 5.1, 5.2, 5.4, 5.5, 5.6, 5.7, 5.8, 6.0, 6.1, 6.2, 6.4, 6.5, 6.6, 6.8, 6.9, 7.0, 7.2, 7.4, 7.5, 7.6, 7.7, 7.8, 7.9, 8.0, 8.1, 8.2, 8.3, 8.4, 8.5, 8.6, 8.8, 8.9, 9.0, 9.2, 9.4, 9.5, 9.6, 9.7, 9.8, 9.9, 10.0 μg/kg/hr, or in any range with any of these values as lower or upper values, such as 1.2 to 2.8 μg/kg/hr, 2.6 to 7.4 μg/kg/hr, 3.4 to 5.8 μg/kg/hr, 4.2 to 8.6 μg/kg/hr, 1.0 to 3.0 μg/kg/hr, 2.8 to 3.6 μg/kg/hr, 1.0 to 4 μg/kg/hr, 1.2 to 4 μg/kg/hr, 1.4 to 4 μg/kg/hr, 1.6 to 4 μg/kg/hr, 1.8 to 4 μg/kg/hr, 2.0 to 4 μg/kg/hr, 2.2 to 4 μg/kg/hr, 2.4 to 4 μg/kg/hr, 2.6 to 4 μg/kg/hr, 2.7 to 4 μg/kg/hr, 2.8 to 4 μg/kg/hr, 1.0 to 5 μg/kg/hr, 1.2 to 5 μg/kg/hr, 1.4 to 5 μg/kg/hr, 1.6 to 5 μg/kg/hr, 1.8 to 5 μg/kg/hr, 2.0 to 5 μg/kg/hr, 2.2 to 5 μg/kg/hr, 2.4 to 5 μg/kg/hr, 2.6 to 5 μg/kg/hr, 2.7 to 5 μg/kg/hr, 2.8 to 5 μg/kg/hr, 3.0 to 5 μg/kg/hr, 3.2 to 5 μg/kg/hr, 3.4 to 5 μg/kg/hr, 3.6 to 5 μg/kg/hr, 1.0 to 6 μg/kg/hr, 1.2 to 6 μg/kg/hr, 1.4 to 6 μg/kg/hr, 1.6 to 6 μg/kg/hr, 1.8 to 6 μg/kg/hr, 2.0 to 6 μg/kg/hr, 2.2 to 6 μg/kg/hr, 2.4 to 6 μg/kg/hr, 2.6 to 6 μg/kg/hr, 2.7 to 6 μg/kg/hr, 2.8 to 6 μg/kg/hr, 3.0 to 6 μg/kg/hr, 3.2 to 6 μg/kg/hr, 3.2 to 6 μg/kg/hr, 3.4 to 6 μg/kg/hr, 3.6 to 6 μg/kg/hr, 3.8 to 6 μg/kg/hr, 4.0 to 6 μg/kg/hr, 1.0 to 8 μg/kg/hr, 1.2 to 8 μg/kg/hr, 1.4 to 8 μg/kg/hr, 1.6 to 8 μg/kg/hr, 1.8 to 8 μg/kg/hr, 2.0 to 8 μg/kg/hr, 2.2 to 8 μg/kg/hr, 2.4 to 8 μg/kg/hr, 2.6 to 8 μg/kg/hr, 2.7 to 8 μg/kg/hr, 2.8 to 8 μg/kg/hr, 3.0 to 8 μg/kg/hr, 3.2 to 8 μg/kg/hr, 3.4 to 8 μg/kg/hr, 3.6 to 8 μg/kg/hr, 3.8 to 8 μg/kg/hr, 4.0 to 8 μg/kg/hr, 4.2 to 8 μg/kg/hr, 4.4 to 8 μg/kg/hr, 4.6 to 8 μg/kg/hr, 4.8 to 8 μg/kg/hr, 5.0 to 8 μg/kg/hr, 1.0 to 10 μg/kg/hr, 1.2 to 10 μg/kg/hr, 1.4 to 10 μg/kg/hr, 1.6 to 10 μg/kg/hr, 1.8 to 10 μg/kg/hr, 2.0 to 10 μg/kg/hr, 2.2 to 10 μg/kg/hr, 2.4 to 10 μg/kg/hr, 2.6 to 10 μg/kg/hr, 2.7 to 10 μg/kg/hr, 2.8 to 10 μg/kg/hr, 3.0 to 10 μg/kg/hr, 3.2 to 10 μg/kg/hr, 3.4 to 10 μg/kg/hr, 3.6 to 10 μg/kg/hr, 3.8 to 10 μg/kg/hr, 4.0 to 10 μg/kg/hr, 4.2 to 10 μg/kg/hr, 4.4 to 10 μg/kg/hr, 4.6 to 10 μg/kg/hr, 4.8 to 10 μg/kg/hr, and 5.0 to 10 μg/kg/hr 5.2 to 10 μg/kg/hr, 5.4 to 10 μg/kg/hr, 5.6 to 10 μg/kg/hr, 5.8 to 10 μg/kg/hr, 6.0 to 10 μg/kg/hr, and so on. In embodiments, the maintenance dose is initiated at a higher rate and then titrated down. In other embodiments, the maintenance dose is initiated at a lower rate and then increased.

The maintenance dose (IV or oral) can be administered over a duration of up to about 60 minutes to several hours to several days to several months to several years, including 0.5, 1.0, 1.5, 2.0, 2.5, 3.0, 3.5, 4.0, 4.5, 5.0, 5.5, 6.0, 6.5, 7.0, 7.5, 8.0, 9, 10, 11, 12, 13, 14, 15, 16, 17, 18, 19, 20, 21, 22, 23, 24, 36, 48, 72 hours, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or including 1, 2, 3, 4, 5, 6, 7, 8, 9, 10, 11, 12 months, or including 1, 2, 3, 4, 5, 8, or 10 years, or in any range with any of these values as lower and upper values, such as 0.5 to 1.5 hours, 1 to 2 hours, 1 to 3 hours, 1 to 4 hours, 1 to 5 hours, 1 to 6 hours, 1 to 10 hours, 1 to 18 hours, 1 to 24 hours, 1 to 36 hours, 1 to 48 hours, 1 to 72 hours, 1 to 168 hours, 2 to 3 hours, 2 to 4 hours, 2 to 5 hours, 2 to 6 hours, 2 to 8 hours, 2 to 10 hours, 2 to 18 hours, 2 to 24 hours, 2 to 36 hours, 2 to 48 hours, 2 to 72 hours, 2 to 168 hours, 3 to 4 hours, 3 to 5 hours, 3 to 6 hours, 3 to 10 hours, 3 to 18 hours, 3 to 24 hours, 3 to 36 hours, 3 to 48 hours, 3 to 72 hours, 3 to 168 hours, 4 to 8 hours, 4 to 10 hours, 4 to 18 hours, 4 to 24 hours, 4 to 36 hours, 4 to 48 hours, 4 to 72 hours, 4 to 168 hours, 6 to 8 hours, 6 to 10 hours, 6 to 18 hours, 6 to 24 hours, 6 to 36 hours, 6 to 48 hours, 6 to 72 hours, 6 to 168 hours, 8 to 10 hours, 8 to 18 hours, 8 to 24 hours, 8 to 36 hours, 8 to 48 hours, 8 to 72 hours, 8 to 168 hours, 12 to 24 hours, 12 to 72 hours, 12 to 168 hours, 24 to 72 hours, 1 to 7 days, 1 to 10 days, 1 to 14 days, 7 to 30 days, 1 to 3 months, 2 to 6 months, 1 to 5 years, and so on.

In embodiments, one or more IV or oral maintenance dose is administered, with a first maintenance dose being administered up to about 12 hours after the conclusion of the administration of the IV loading dose, such as immediately after the conclusion of the administration or up to about 6 hours after, such as about 0.5 hours, 0.75 hours, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, or 5.75 hours after administration of the IV loading dose. In embodiments, the first maintenance dose is administered about 1 hour to about 12 hours after conclusion of the administration of the loading dose, such as about 2 hours to about 11 hours, about 3 hours to about 10 hours, about 4 hours to about 9 hours, about 5 hours to about 8 hours, or about 6 hours to about 7 hours. In embodiments, the subject/patient is administered the first maintenance dose after being released from the hospital or other facility that was providing the monitoring, but the patient/subject can still be monitored in a similar way, such as by using a portable/wearable ECG monitoring system. In other embodiments, the patient/subject is not monitored using a portable/wearable ECG monitoring system after release from the hospital or other facility that was providing monitoring. In embodiments, a first maintenance dose is administered before the patient/subject is released from the hospital or other facility that was providing the monitoring.

The maintenance dose can be intravenously infused into a central or peripheral vein of the patient. In other embodiments, the maintenance dose can be given orally. In an embodiment, the maintenance dose is initiated as an IV infusion, then transitioned to oral administration. In embodiments, multiple maintenance doses are given, wherein one or more maintenance dose(s) is administered as an IV infusion and one or more maintenance dose(s) is administered orally. In other embodiments, all maintenance doses are administered orally. In other embodiments, all maintenance doses are administered intravenously.

In embodiments, the one or more oral dose is given in an amount of about 500 μg, about 250 μg, or about 125 μg. In embodiments, more than one oral dose amount is given to a particular patient (for example, a patient receives one or more oral dose at 250 μg and a subsequent higher oral dose of 500 μg or a subsequent lower oral dose of 125 μg, or any combination thereof).

The patient can be monitored by electrocardiogram (ECG) for normal sinus rhythm and/or ventricular arrhythmias or ventricular tachycardia and the maintenance dose rate adjusted upward until normal sinus rhythm is reached or adjusted downward if ventricular arrhythmias, ventricular tachycardia, or other side effects typically associated with dofetilide administration, such as dizziness occur. The maintenance dose can be adjusted for patient QT interval (or QTc) as described below. The maintenance dose rate can be adjusted for patient creatinine clearance as described below.

The flow rate of the intravenous dose can be adjusted based on the concentration of dofetilide in the intravenous formulation and the body weight of the patient to achieve a specific maintenance dose rate in μg/kg/hr. The maintenance dose rate can be targeted to reach a particular therapeutic, steady-state plasma concentration based on established pharmacokinetic parameters of dofetilide such as half-life and clearance. As can be appreciated, the cumulative maintenance dose is a product of the rate of intravenous infusion in μg/kg/hr multiplied by the infusion duration in hours.

In embodiments, the IV loading dose is administered in a hospital or other facility that can provide cardiac resuscitation and continuous electrocardiographic monitoring and a first maintenance dose and subsequent maintenance doses are administered by the patient after discharge from the facility/hospital, but may still be under monitoring, such as self-monitoring. In other embodiments, the IV loading dose and a first maintenance dose are administered in a hospital or other facility that can provide cardiac resuscitation and continuous electrocardiographic monitoring and subsequent maintenance doses are administered by the patient after discharge from the facility/hospital.

In embodiments, subsequent maintenance doses are given after the first maintenance dose. In embodiments, the subsequent maintenance doses are administered at intervals of about 12 hours, about 24 hours, or about 48 hours. In embodiments, the subject/patient is administered subsequent maintenance dose(s) after being released from the hospital or other facility capable of providing cardiac resuscitation and continuous electrocardiographic monitoring.

In embodiments, the IV loading dose and/or maintenance dose(s) are given in amounts and at time intervals such that the patient achieves or is capable of achieving Cₘₐₓ ss within about 8 hours, or even within about 2 hours, of the start of the administration of the IV loading dose. In embodiments, Cₘₐₓ ss is achieved before administration of the first maintenance dose.

In embodiments, the IV loading dose is given in an amount such that the patient experiences a Cₘₐₓ that is at least about 80% of the Cₘₐₓ ss for the patient's specific dosing protocol within about 2 hours of initiation of the loading dose. In embodiments, following the IV loading dose, such as before the first oral dose, the patient experiences or is capable of experiencing a Cₘₐₓ that is at least about 85% of the Cₘₐₓ ss, such as about 87%, 90%, 92%, 95%, 97% or 99% of the Cₘₐₓ ss for the patient's specific dosing protocol.

QT Interval Monitoring

Proper dosing for dofetilide can be ascertained by monitoring the QT interval, or the heart rate corrected QT (the QTc), during and/or after IV infusion of the loading and/or maintenance dose(s), and thus avoid the development of life-threatening ventricular tachycardias such as Torsades de Pointes. Patients can be monitored by electrocardiogram (ECG) and a baseline QT or QTc can be measured. A baseline QT or QTc exceeding a certain threshold, such as 440 msec (or 500 msec for patients with ventricular conduction abnormalities), may exclude some patients from dofetilide treatment (see US Patent Application Publication No. 20190388371A1). For patients that qualify for treatment, the QT and/or QTc can be monitored during infusion of the loading and/or maintenance dose, and such dose can be adjusted downward or ceased if the patient's QT or QTc becomes too high, either over a QT or QTc threshold or a change exceeding a patient's baseline QT or QTc. In one implementation, the QT or QTc threshold is 500 msec. In various implementations, the change exceeding baseline QTc can be 5%, 10%, 15%, or 20% over baseline QTc. The QT or QTc can be measured at regular intervals, such as every 10, 15, 20, 30, 45, or 60 minutes during treatment. In some situations, such as if the patient's heart rate is less than 60 bpm, the uncorrected QT interval can be used. Remote ECG monitoring, such as Holter monitoring or event monitoring, may be indicated for long term monitoring of QT interval or QTc. In embodiments, the patient/subject is not monitored using a portable/wearable ECG monitoring system after release from the hospital or other facility that was providing monitoring.

In embodiments, one or more of the maintenance doses is selected based on a patient's change in QT or QTc (for example, a patient that experiences an increase in QTc of less than 15% of baseline after receiving the loading dose may receive a higher maintenance dose than a patient that experiences an increase in QT or QTc of 15% or greater after receiving the loading dose). In embodiments, the maintenance dose is changed after administration of a previous maintenance dose due to a change in the patient's QTc (for example, a patient given an oral dose of 250 μg experiences an increase in QTc of 15% or greater, and the next oral dose can be lowered to 125 μg).

Creatinine Clearance

Because elimination of dofetilide is primarily renal, renal impairment can potentially lead to toxic serum concentrations. As such, it is desirable to measure patient creatinine clearance prior to initiating a dofetilide dosing regimen in order to gauge kidney function. Serum samples can be taken from the patient prior to treatment with dofetilide and serum creatinine measured according the following formulas:

Creatinine clearance (male)=[(140−Age)×Body Wt (kg)]/[72×serum creatinine (mg/dL)]

Creatinine clearance (female)=Creatine clearance (male)×0.85

The loading dose and maintenance dose can be adjusted according to creatinine clearance as provided in Table I.

TABLE I

| Creatinine | | |
|---|---|---|
| Clearance (mL/min) | Loading Dose | Maintenance Dose |
| >60 | High | High |
| 40-60 | Medium | Medium |
| 20-<40 | Low | Low |
| <20 | Dofetilide not indicated | Dofetilide not indicated |

In other embodiments, a patient/subject with a lower creatinine clearance may receive a lower loading dose or maintenance dose than a patient with a higher creatinine clearance, or in some cases a patient/subject with a higher creatinine clearance may receive a lower loading dose or maintenance dose than a patient with a lower creatinine clearance. In some cases, for example, the amount of dofetilide, whether by IV or an oral dose, appropriate to administer to a subject with a CrCl of 20-<40 mL/min or 40-60 mL/min can be higher than is appropriate for a subject with a CrCl of >60 mL/min.

Indications

In embodiments, dofetilide is administered intravenously according to any of the above loading and/or maintenance doses and durations to treat or prevent such conditions as atrial fibrillation or flutter (sustained or intermittent), paroxysmal atrial fibrillation, ventricular tachycardia, ventricular fibrillation, paroxysmal supraventricular tachycardia, heart failure, coronary artery disease, pulmonary artery hypertension, and the like, and for situations where the patient is unable to take an anti-arrhythmic by mouth (NPO). The specific intravenous loading dose and intravenous maintenance dose rate protocols would be selected based upon the patient's condition, baseline and in-treatment QT interval or QTc, as well as the patient's creatinine clearance as described herein. Other factors affecting selection of the loading and maintenance dose include patient body weight. Oral maintenance doses of dofetilide can be administered to the patient according to available oral dosages (125 μg, 250 μg, 500 μg, typically given twice daily), QTc, creatinine clearance, body weight, and other factors, as described by U.S. Patent Application Publication No. 20190388371A1). Situations where IV loading and/or maintenance doses are appropriate include those in which the patient cannot take oral administration (NPO), or for gastrointestinal conditions resulting in poor absorption, recovery from GI surgery, and/or intensive care.

Dofetilide administration according to embodiments of the invention is administered to patients naïve to dofetilide treatment and/or to patients currently receiving dofetilide therapy and/or to patients having previously received dofetilide.

In embodiments, the dofetilide dose (including the loading dose and/or maintenance dose, whether IV or oral) is determined by patient characteristics including body weight, sex, and/or creatinine clearance.

The following Examples are provided to illustrate various protocols for administering dofetilide.

### EXAMPLE 1

A physician determines the patient should be initiated on (or escalated to, if the patient had been administered a prior lower dose) a dofetilide dosing protocol of 500 μg BID. The patient is admitted to a hospital, or other facility capable of providing cardiac resuscitation and continuous electrocardiographic monitoring, and can be currently in sinus rhythm, recently cardioverted to sinus rhythm, or administered dofetilide to convert the patient to sinus rhythm. The patient's creatinine clearance is measured/determined. The patient is connected to an electrocardiograph, a baseline QT interval (or QTc) is measured/determined, and treatment is initiated with an IV loading dose of dofetilide in an amount of up to about 750 μg infused over a period of about 1 hour. The amount of the dofetilide IV dose is based on the creatinine clearance of the patient, such as an amount of dofetilide in the range of 400-450 μg (for a CrCl of >60 mL/min), 425-550 μg (for a CrCl in the range of 40-60 mL/min), or 440-475 μg (for a CrCl in the range of 20 to <40 mL/min).

The patient's QT interval is measured/determined prior to start of the IV and is monitored every 15 minutes or so during the IV loading dose administration, optionally along with heart rate (HR) and blood pressure (BP). The patient's QTc is calculated from the QT measurement. If the patient's baseline (i.e., prior to dofetilide administration) QT interval or baseline QTc is greater than 440 msec (or 500 msec in patients with ventricular conduction abnormalities), then dofetilide is contraindicated. During the IV loading dose, if the QT or QTc increases by greater than 15% of the patient's baseline QT or baseline QTc (or is greater than 500 msec or 550 msec in patients with ventricular conduction abnormalities), then the IV dofetilide is discontinued or a subsequent lower dose is considered or administered. If after any subsequent IV or oral dose the QT or QTc increases to greater than 500 msec (or 550 msec in patients with ventricular conduction abnormalities) or increases by greater than 15% of the patient's baseline QT or baseline QTc, then dofetilide is discontinued.

Up to 12 hours after completion of the loading dose, the patient receives an oral maintenance dose of 500 μg of dofetilide. The patient's QT or QTc is monitored at a selected interval, such as every hour, after the administration of the oral maintenance dose. If after the oral maintenance dose the patient's QT or QTc is greater than 500 msec (or 550 msec in patients with ventricular conduction abnormalities) or if the QT or QTc increases by greater than 15% of the patient's baseline QT or QTc, then the next oral maintenance dose is reduced to 250 μg of dofetilide and/or is administered 24 to 48 hours after the last oral dose.

For the situation where QT or QTc is within an acceptable range, either after the IV dose or after the first or any subsequent maintenance dose of dofetilide, the patient can be released from the hospital/facility to continue dofetilide treatment outside the hospital/facility. For the situation where (i) the patient's QT or QTc after administration of the first IV maintenance dose is greater than 500 msec (or 550 msec in patients with ventricular conduction abnormalities) or increases by greater than 15% of the patient's baseline QT or QTc, (ii) the patient's maintenance dose is reduced further, and (iii) the QT or QTc for the further reduced maintenance dose (or any subsequent maintenance dose) is observed to be greater than 500 msec (or 550 msec in patients with ventricular conduction abnormalities) or increases by greater than 15% of the patient's baseline QT or QTc, additional subsequent maintenance doses can be further reduced to 125 μg, and/or dofetilide treatment is discontinued.

### Example 2

A patient in need of dofetilide treatment is admitted to a hospital or other facility capable of providing cardiac resuscitation and continuous electrocardiographic monitoring. The patient's creatinine clearance is determined. The patient is connected to an electrocardiograph, and an initial (or baseline) QT or QTc interval is determined to be not greater than 440 msec (or 500 msec in patients with ventricular conduction abnormalities). Treatment is initiated for a dofetilide dosing protocol of 250 μg BID with an IV loading dose of up to 375 μg of dofetilide infused over a period of up to 1 hour. The amount of the dofetilide IV dose is based on the creatinine clearance of the patient, such as an amount of dofetilide in the range of 175-200 μg (for a CrCl of >60 mL/min), 190-300 μg (for a CrCl in the range of 40-60 mL/min), or 210-240 μg (for a CrCl in the range of 20 to <40 mL/min). The loading dose may be administered by way of two or several smaller IV doses. The patient's QT or QTc interval is measured/determined every 15 minutes or so during the IV loading dose administration along with HR and BP.

During or following administration of the IV loading dose, the patient's QT or QTc interval is determined to be greater than 500 msec (or 550 msec for patients with ventricular conduction abnormalities) or increases by greater than 15% of the patient's baseline QT or QTc. A reduced maintenance dose is administered to the patient up to 12 hours after completion of the IV loading dose or dofetilide administration is discontinued. The reduced maintenance dose is administered as an oral dose of 125 μg (for the previous target maintenance dose of 250 μg) or 250 μg (for the previous target maintenance dose of 500 μg). Dofetilide administration can be discontinued for the previous target maintenance dose of 125 μg where the patient is found not to tolerate dofetilide. The patient's QT or QTc interval is monitored following administration of the oral maintenance dose. The patient's QT or QTc interval no longer exceeds 500 msec (or 550 msec in patients with ventricular conduction abnormalities) and the ΔQT or ΔQTc is not greater than 15% of the patient's initial QT or QTc. The patient is discharged with instructions to self-administer the reduced oral maintenance doses of 125 μg or 250 μg, as appropriate, every 12 to 48 hours.

### Example 3

An example anti-arrhythmic/dofetilide treatment protocol described herein. The patient is admitted to a hospital or other facility capable of providing cardiac resuscitation and continuous electrocardiographic monitoring. The patient's creatinine clearance is determined to be between 20 and <40 mL/min. The patient is connected to an electrocardiograph, and an initial QT or QTc interval is determined to be not greater than 440 msec (or 500 msec in patients with ventricular conduction abnormalities). Treatment is initiated with an IV loading dose of up to 12 μg/kg infused over a period of up to 1 hour, such in the range of about 105-115 μg (for a target maintenance dose of 125 μg), 210-240 μg (for a target maintenance dose of 250 μg), or 440-475 μg (for a target maintenance dose of 500 μg). The loading dose may be administered by way of two or more smaller IV doses. The patient's QT or QTc interval is measured/determined about every 15 minutes of so during the IV loading dose administration along with HR and BP.

During and following administration of the loading dose, the patient's QT or QTc interval is determined to be not greater than 500 msec (or 550 msec for patients with ventricular conduction abnormalities) and ΔQT or ΔQTc is not greater than 15% of the patient's initial QT or QTc. A maintenance dose is administered to the patient up to 12 hours after completion of the IV loading dose. After the IV dose, the patient can be discharged from the hospital/facility with instructions to administer oral maintenance doses of 125 μg, 250 μg, or 500 μg, accordingly, every 12 to 48 hours. Alternatively, the first oral maintenance dose can be given to the patient in the hospital/facility and then the patient can be discharged with instructions to administer subsequent oral maintenance doses of 125 μg, 250 μg, or 500 μg, accordingly, every 12 to 48 hours. The patient's QT or QTc interval is monitored following administration of the oral maintenance dose and any subsequent oral maintenance doses and if the patient's QT or QTc interval still does not exceed 500 msec (or 550 msec in patients with ventricular conduction abnormalities) and is not greater than 15% of the patient's initial QT or QTc, the dofetilide treatment can be continued.

### Example 4

An example dofetilide treatment protocol is described herein. The patient is admitted to a hospital or other facility capable of providing cardiac resuscitation and continuous electrocardiographic monitoring to initiate treatment. The patient's creatinine clearance is determined to be 40-60 mL/min. The patient is connected to an electrocardiograph, and an initial QT or QTc interval is measured/determined. Treatment is initiated with an IV loading dose based on an oral target dose of 500 μg, which IV dose is administered in an amount in the range of ±50% of the oral target dose, such as an amount of dofetilide in the range of about 425-550 μg, and is infused over a period of up to 1 hour. The patient's QT or QTc interval is measured/determined every 15 minutes or so during the IV loading dose administration along with HR and BP.

During and following administration of the loading dose, the patient's QT or QTc interval is determined to be not greater than 500 msec (or 550 msec for patients with ventricular conduction abnormalities) and the ΔQT or ΔQTc is not greater than 15% of the patient's initial QT or QTc. An oral or IV maintenance dose is administered to the patient in an amount of 500 μg. The patient's QT or QTc interval is monitored following administration of the oral or IV maintenance dose. The patient would be expected to experience a Cₘₐₓ ss within 8 hours of the initiation of the IV loading dose. The patient's QTc interval still does not exceed 500 msec (or 550 msec for patients with ventricular conduction abnormalities) and the ΔQT or ΔQTc is not greater than 15% of the patient's initial QT or QTc. The patient would be discharged with instructions to administer subsequent oral maintenance doses of 500 μg every 12 hours.

### Example 5

An example anti-arrhythmic/dofetilide treatment protocol is described herein. The patient is admitted to a hospital or other facility capable of providing cardiac resuscitation and continuous electrocardiographic monitoring to initiate treatment. The patient's creatinine clearance is determined to be between 40 and 60 mL/min. The patient is connected to an electrocardiograph, and an initial QT or QTc interval is determined. Treatment is initiated with an IV loading dose based on an oral target of 250 μg, which is administered in an amount in the range of +50% of the oral target dose, such as in an amount in the range of 190-300 μg, and is infused over a period of up to about 1 hour. The patient's QT or QTc interval is measured/determined during the IV loading dose administration, optionally every 15 minutes or so, along with HR and BP.

During and following administration of the IV loading dose, the patient's QT or QTc interval is determined to be not greater than 500 msec (or 550 msec in patient with ventricular conduction abnormalities) and the ΔQT or ΔQTc is not greater than 15% of the patient's initial QTc. A maintenance dose is administered to the patient as an IV or oral dose of 250 μg. The patient's QT or QTc interval is monitored during and following administration of the IV or oral maintenance dose. The patient's QT or QTc interval still does not exceed 500 msec (or 550 msec in patients with ventricular conduction abnormalities) and the ΔQT or ΔQTc is not greater than 15% of the patient's initial QT or QTc. The patient is discharged from the hospital/facility with instructions to be administered subsequent IV or oral maintenance doses of up to 250 μg every 12 to 48 hours.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of administering dofetilide comprising:
administering to a subject an intravenous (IV) dosage of dofetilide, wherein the subject is in a facility capable of providing cardiac resuscitation and continuous electrocardiographic monitoring, and wherein the IV dosage is administered:
(a) in an amount of about 62.5-187.5 μg;
(b) in an amount of about 125-375 μg; or
(c) in an amount of about 250-750 μg; and
after completion of the administering of the IV dosage, administering a maintenance dose of dofetilide;
optionally administering a subsequent maintenance dose of dofetilide at least once at a selected interval, optionally after the subject is discharged from the facility capable of providing cardiac resuscitation and continuous electrocardiographic monitoring.

2. The method of claim 1, wherein the IV dosage of dofetilide and the maintenance dose are administered to the subject in a facility capable of providing cardiac resuscitation and continuous electrocardiographic monitoring, and the subsequent maintenance dose of dofetilide, if administered, is administered after the subject is discharged from the facility capable of providing cardiac resuscitation and continuous electrocardiographic monitoring.

3. The method of claim 1, wherein the IV dosage of dofetilide is administered as a single IV dosage over a period of about 1-5 hours, and the maintenance dose of dofetilide is administered at least 1 hour after completion of the administration of the IV dosage of dofetilide.

4. The method of claim 1, wherein:
the maintenance dose of dofetilide is an intravenous maintenance dose and/or an oral maintenance dose; and
the subsequent maintenance dose of dofetilide, if administered, is a subsequent intravenous maintenance dose and/or a subsequent oral maintenance dose comprising an amount of dofetilide that is the same or a different amount of dofetilide as in the maintenance dose.

5. The method of claim 4, wherein the maintenance dose and/or the subsequent maintenance dose, if administered, comprises an amount of dofetilide that is less than the IV dosage, and/or the subsequent maintenance dose, if administered, comprises an amount of dofetilide that is less than the maintenance dose.

6. The method of claim 1, wherein the IV dosage comprises dofetilide in an amount in the range of about 50% to about 110% of the amount of dofetilide in the maintenance dose.

7. The method of claim 6, wherein the maintenance dose and/or the subsequent maintenance dose, if administered, is an oral maintenance dose of dofetilide in an amount of 125 μg, 250 μg, or 500 μg.

8. The method of claim 6, wherein the maintenance dose and/or the subsequent maintenance dose, if administered, is an intravenous maintenance dose of dofetilide in an amount in the range of about 62.5 μg-750 μg.

9. The method of claim 1, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

10. The method of claim 1, further comprising:
measuring a baseline QT interval and/or determining a baseline QTc interval for the subject before administering the IV dosage;
determining one or more QT interval(s) or QTc interval(s) after administration of the maintenance dose;
if at least one of the one or more QT or QTc intervals determined after the maintenance dose is more than 500 msec or increases greater than 15% from the baseline QT or QTc interval, refraining from administering a subsequent maintenance dose of dofetilide.

11. The method of claim 1, wherein the IV dosage of dofetilide is administered:
in a first higher amount for the subject who is renally impaired; and
in a second lower amount for the subject who is non-renally impaired.

12. The method of claim 1, wherein the amount of dofetilide for any one or more or all doses is below 1.8 μg/kg.

13. The method of claim 1, wherein the amount of dofetilide for any one or more or all doses is above 3 μg/kg.

14. The method of claim 1, wherein the amount of dofetilide for any one or more or all doses is an amount in the range of about: 85-100 μg, 90-150 μg, 105-115 μg, 175-200 μg, 190-300 μg, 210-240 μg, 400-450 μg, 425-550 μg, or 440-475 μg.

15. The method of claim 14, wherein the IV dosage comprises an amount of dofetilide based on a creatinine clearance of the subject as follows:

| | Intravenous Dosage of Dofetilide | | |
|---|---|---|---|
| | | | Subject's |
| Maintenance | Subject's | Subject's | CrCl 20 mL/min |
| Dose | CrCl >60 mL/min | CrCl 40-60 mL/min | to <40 mL/min |
| 125 μg | 85-100 μg | 90-150 μg | 105-115 μg |
| 250 μg | 175-200 μg | 190-300 μg | 210-240 μg |
| 500 μg | 400-450 μg | 425-550 μg | 440-475 μg |

16. A method of administering dofetilide, comprising:
(A) before administering an IV dosage of dofetilide to a subject, determining a creatinine clearance (CrCl) of the subject, and selecting an amount of dofetilide to administer to the subject based on the creatinine clearance;
(B) administering to the subject the IV dosage of dofetilide comprising the selected amount of dofetilide based on the creatinine clearance and comprising an amount of dofetilide in the range of 0.1-12 μg/kg, and
(C) after the administering of the IV dosage, administering a first maintenance dose of dofetilide to the subject;
(D) optionally administering one or more subsequent maintenance doses of dofetilide at selected interval from the first maintenance dose.

17. The method of claim 16, wherein the IV dosage and the first maintenance dose of dofetilide are administered to the subject in a facility capable of providing cardiac resuscitation and continuous electrocardiographic monitoring, and the subsequent maintenance dose of dofetilide, if administered, is administered after the subject is discharged from the facility capable of providing cardiac resuscitation and continuous electrocardiographic monitoring.

18. The method of claim 16, wherein the first maintenance dose of dofetilide is an intravenous maintenance dose and/or an oral maintenance dose.

19. The method of claim 18, wherein:
the first maintenance dose and/or one or more of the subsequent maintenance doses, if administered, of dofetilide is a higher dose than the IV dosage.

20. The method of claim 18, wherein:
the first maintenance dose and/or one or more of the subsequent maintenance doses, if administered, of dofetilide is a lower dose than the IV dosage.

21. The method of claim 16, wherein the IV dosage comprises an amount of dofetilide in the range of about ±50% of the amount of the first maintenance dose.

22. The method of claim 16, wherein the IV dosage is administered over about 60 minutes.

23. The method of claim 16, wherein the subject is capable of experiencing a dofetilide Cₘₐₓ steady state within about 8 hours of administration of the IV dosage.

24. The method of claim 23, wherein the IV dosage is administered to the subject in an amount and over a period of time such that the dofetilide reaches or is capable of reaching a Cₘₐₓ in the subject that is at least about 70% of a steady state Cₘₐₓ for an oral dosing protocol of 125 μg, 250 μg, or 500 μg dofetilide.

25. The method of claim 16, wherein the IV dosage of dofetilide is administered:
in a first higher amount for the subject with a CrCl of 20 mL/min to <40 mL/min; and
in a second lower amount for the subject with a CrCl of 40 mL/min or greater.
